(19) United States
(12) Patent Application Publication
(10) Pub. No.: US 2014/0290395 A1
(43) Pub. Date: Oct. 2, 2014

(54) Preservation System for Nutritional Substances

(71) Applicant: Eugenio Minvielle, Rye, NY (US)

(72) Inventor: Eugenio Minvielle, Rye, NY (US)

(21) Appl. No.: 14/304,671

(22) Filed: Jun. 13, 2014

Related U.S. Application Data
(62) Division of application No. 13/888,353, filed on May 6, 2013.
Application No. 13/888,353 is a continuation-in-part of application No. 13/485,854, filed on May 31, 2012.
(60) Provisional application No. 61/624,985, filed on Apr. 16, 2012.
(60) Provisional application No. 61/624,948, filed on Apr. 16, 2012.
(60) Provisional application No. 61/624,972, filed on Apr. 16, 2012.

(51) Int. Cl. G01N 33/02

(52) U.S. Cl. CPC G01N 33/02
USPC 73/865.8

(57) ABSTRACT

Disclosed herein is preservation system for nutritional substances. The preservation system obtains information about the nutritional substance to be preserved, senses and measures the external environment to the preservation system, senses and measures the internal environment to the preservation system, senses and measures the state of the nutritional substance, and stores such information throughout the period of preservation. Using this accumulated information, the preservation system can measure, or estimate, changes in nutritional content (usually degradation) during the period of preservation. Additionally, the preservation system can use this information to dynamically modify the preservation system to minimize detrimental changes to the nutritional content of the nutritional substance, and in some cases actually improve the nutritional substance attributes.

### RELATED PATENT APPLICATIONS

This application is a divisional application of U.S. patent application Ser. No. 13/888,353 filed on May 6, 2013, which is a continuation-in-part of U.S. patent application Ser. No. 13/485,854, filed May 31, 2012, which claims benefit under 35 U.S.C. 119(e) of U.S. Provisional Patent Application Ser. No. 61/624,948 filed Apr. 16, 2012; U.S. Provisional Patent Application Ser. No. 61/624,972, filed Apr. 16, 2012; and U.S. Provisional Patent Application 61/624,985, filed Apr. 16, 2012, the contents of which are incorporated herein by reference in their entirety.

### FIELD OF THE INVENTION

The present inventions relate to collection, transmission, creation and use of information regarding the preservation of nutritional substances.

### BACKGROUND OF THE INVENTION

Nutritional substances are traditionally grown (plants), raised (animals) or synthesized (synthetic compounds). Additionally, nutritional substances can be found in a wild, non-cultivated form, which can be caught or collected. While the collectors and creators of nutritional substances generally obtain and/or generate information about the source, history, caloric content and/or nutritional content of their products, they generally do not pass such information along to the users of their products. One reason is the nutritional substance industries have tended to act like “silo” industries. Each group in the food and beverage industry: growers, packagers, processors, distributors, retailers, and preparers work separately, and either shares no information, or very little information, between themselves. There is generally no consumer access to, and little traceability of, information regarding the creation and/or origin, preservation, processing, preparation, or consumption of nutritional substances. In particular, there is no information available to a consumer, at the moment the consumer wants to know, regarding changes (typically degradation) in nutritional, organoleptic, or aesthetic values of nutritional substances or regarding residual nutritional, organoleptic, or aesthetic values of the nutritional substance. Further, there is no information available to the consumer regarding changes in nutritional, organoleptic, or aesthetic values of nutritional substances or regarding residual nutritional, organoleptic, or aesthetic values of the nutritional substance after they have been conditioned, and no way for the consumer to know what conditioning protocol will achieve the nutritional, organoleptic, or aesthetic values he desires. It would be desirable for such information be available to the consumers of nutritional substances at any desired moment, as well as all participants in the food and beverage industry—the nutritional substance supply system.

While the nutritional substance supply system has endeavored over the last 50 years to increase the caloric content of nutritional substances produced (which has help reduce starvation in developing countries, but has led to obesity problems in developed countries), maintaining, or increasing, the nutritional content of nutritional substances has been a lower priority. Caloric content refers to the energy in nutritional substances, commonly measured in calories. The caloric content could be represented as sugars and/or carbohydrates in the nutritional substances. The nutritional content, also referred to herein as nutritional value, of foods and beverages, as used herein, refers to the non-caloric content of these nutritional substances which are beneficial to the organisms which consume these nutritional substances. For example, the nutritional content of a nutritional substance could include vitamins, minerals, proteins, and other non-caloric components which are necessary, or at least beneficial, to the organism consuming the nutritional substances.

While there has recently been greater attention by consumer organizations, health organizations and the public to the nutritional content of foods and beverages, the food and beverage industry has been slow in responding to this attention. Today's innovation, research and scientific advances of food and Beverage industry have been primarily focused on producing more volume and preserving nutritional substances longer to supply the needs of our population. More over the industry has developed in silos increasingly adding dyes, preservatives, artificial flavors, enhancers, artificial sweeteners, pesticides, hormones, antibiotics, and other additives to fulfill this role. In particular since 1940, over 75,000 artificial chemicals have been created and many appear in food products, such as blueberry muffins, “diet” sodas.

One reason for this may be that since the food and beverage industry operates as silos of those who create nutritional substances, those who preserve and transport nutritional substances, those who transform nutritional substances, and those who finally prepare the nutritional substances for consumption by the consumer, there has been no system wide coordination or management of nutritional content. While each of these silo industries may be able to maintain or increase the nutritional content of the foods and beverages they handle, each silo industry has only limited information and control of the nutritional substances they receive, and the nutritional substances they pass along. An interactive system and data base, including user-friendly dynamic nutritional substance labeling allowing consumers, and any other member or other member of the nutritional substance supply system, to access creation and origin information for nutritional substances as well as information regarding changes in nutritional, organoleptic, or aesthetic values of nutritional substances, at any moment during the life-cycle of the nutritional substance up to the moment of consumption, would offer great value to the nutritional substance supply system.

As consumers better understand their need for nutritional substances with higher nutritional content, they will start demanding that the food and beverage industry offer products which include higher nutritional content, and/or at least information regarding nutritional content of such products. In fact, consumers are already willing to pay higher prices for higher nutritional content. This can be seen at high-end grocery stores which offer organic, minimally processed, fresh, non-adulterated nutritional substances. Further, as societies and governments seek to improve their constituents' health and lower healthcare costs, incentives and/or mandates will be given to the food and beverage industry to track, maintain, and/or increase the nutritional content of nutritional substances they handle. There will be a need, not only within each food and beverage industry silo to maintain or improve the nutritional content of their products, but an industry-wide solution to allow the management of nutritional content across the entire cycle from creation to consumption. In order to manage the nutritional content of nutritional substances across the entire cycle from creation to consumption, the nutritional substance industry will need to identify, track, measure, estimate, preserve, transform, condition, and record nutritional content for nutritional substances. Providing nutritional substances with user friendly dynamic nutritional substance labeling facilitating this type of information connectivity and access will be a key in a system capable of such functionality. Of particular importance is the measurement, estimation, and tracking of changes to the nutritional content, also referred to herein as ΔN, of a nutritional substance from creation to consumption. This ΔN information could be used, not only by the consumer in selecting particular nutritional substances to consume, but could be used by the other food and beverage industry silos, including creation, preservation, transformation, and conditioning, to make decisions on how to create, handle and process nutritional substances. Additionally, those who sell nutritional substances to consumers, such as restaurants and grocery stores, could communicate perceived qualitative values of the nutritional substance in their efforts to market and position their nutritional substance products. Further, a determinant of price of the nutritional substance could be particular nutritional, organoleptic, or aesthetic values, and if changes to those values, also referred to herein as ΔN, are perceived as desirable. For example, if a desirable value has been maintained, improved, or minimally degraded, it could be marketed as a premium product.

For example, the grower of sweet corn generally only provides basic information as the variety and grade of its corn to the packager, who preserves and ships the corn to a producer for use in a ready-to-eat dinner. The packager may only tell the producer that the corn has been frozen as loose kernels of sweet corn. The producer may only provide the consumer with rudimentary instructions how to cook or reheat the ready-to-eat dinner in a microwave oven, toaster oven or conventional oven, and only tell the consumer that the dinner contains whole kernel corn among the various items in the dinner. Finally, the consumer of the dinner will likely not express opinions on the quality of the dinner, unless it was an especially bad experience, where the consumer might contact the producer's customer support program to complain. Very minimal, or no, information on the nutritional content of the ready-to-eat dinner is passed along to the consumer. The consumer knows essentially nothing about changes (generally a degradation, but could be a maintenance or even an improvement) to the nutritional content, ΔN, of the sweet corn from creation, processing, packaging, cooking, preservation, preparation by consumer, and finally consumption by the consumer. Unfortunately, today consumers have no way to access information regarding the extent to which nutritional substances have degraded at any moment during their life-cycle, including no information regarding how a nutritional substance will degrade during conditioning. Further, they have no way to access information regarding how to condition a nutritional substance in order to achieve desired nutritional, organoleptic, or aesthetic values. An interactive system and data base including user friendly dynamic nutritional substance labeling allowing consumers to access such information for nutritional substances would offer great value to the nutritional substance supply system.

Consumers' needs are changing as consumers are demanding healthier foods, such as “organic foods.” Customers are also asking for more information about the nutritional substances they consume, such as specific characteristics' relating not only to nutritional content, but to allergens or digestive intolerances. For example, nutritional substances which contain lactose, gluten, nuts, dyes, etc. need to be avoided by certain consumers. However, the producer of the ready-to-eat dinner, in the prior example, has very little information to share other than possibly the source of the elements of the ready-to-eat dinner and its processing steps in preparing the dinner. Generally, the producer of the ready-to-eat dinner does not know the nutritional content and organoleptic state and aesthetic condition of the product after it has been reheated or cooked by the consumer, cannot predict changes to these properties, ΔN, and cannot inform a consumer of this information to enable the consumer to better meet their needs. For example, the consumer may want to know what proportion of desired organoleptic properties or values, desired nutritional content or values, or desired aesthetic properties or values of the corn in the ready-to-eat dinner remain after cooking or reheating, and the change in the desired nutritional content or values, the desired organoleptic properties or values, or the desired aesthetic properties or values, ΔN, (usually a degradation, but could be a maintenance or even improvement). There is a need to preserve, measure, estimate, store and/or transmit information regarding such nutritional, organoleptic, and aesthetic values, including changes to these values, ΔN, throughout the nutritional substance supply system.

The caloric and nutritional content information for a prepared food that is provided to the consumer is often minimal. For example, when sugar is listed in the ingredient list, the consumer may not receive any information about the source of the sugar, which can come from a variety of plants, such as sugarcane, beets, or corn, which will affect its nutritional content. Conversely, some nutritional information that is provided to consumers is so detailed, the consumer can do little with it. For example, this list of ingredients is from a nutritional label on a consumer product: Vitamins—A 355 IU 7%, E 0.8 mg 4%, K 0.5 mcg, 1%, Thiamin 0.6 mg 43%, Riboflavin 0.3 mg 20%, Niacin 6.0 mg 30%, B6 1.0 mg 52%, Foliate 31.5 mcg 8%, Pantothenic 7%; Minerals Calcium 11.6 1%, Iron 4.5 mg 25%, Phosphorus 349 mg 35%, Potassium 476 mg 14%, Sodium 58.1 mg 2%, Zinc 3.7 mg 24%, Copper 0.5 mg 26%, Manganese 0.8 mg 40%, Selenium 25.7 mcg 37%; Carbohydrate 123 g, Dietary fiber 12.1 g, Saturated fat 7.9 g, Monosaturated Fat 2.1 g, Polysaturated Fat 3.6 g, Omega 3 fatty acids 108 g, Omega 6 fatty acids 3481, Ash 2.0 g and Water 17.2 g. (%=Daily Value). There is a need for dynamic labeling of nutritional substances in order to provide information about nutritional substances in a meaningful manner. Such information needs to be presented in a manner that meets the specific needs of a particular consumer. For example, consumers with a medical condition, such as diabetes, would want to track specific information regarding nutritional values associated with sugar and other nutrients in the foods and beverages they consume, and would benefit further from knowing changes in these values or having tools to quickly indicate or estimate these changes in a retrospective, current, or prospective fashion.

If fact, each silo in the food and beverage industry already creates and tracks some information, including caloric and nutritional information, about their product internally. For example, the farmer who grew the corn knows the variety of the seed, condition of the soil, the source of the water, the fertilizers and pesticides used, and can measure the caloric and nutritional content at creation. The packager of the corn knows when it was picked, how it was transported to the packaging plant, how the corn was preserved and packaged before being sent to the ready-to-eat dinner producer, when it was delivered to the producer, and what degradation to caloric and nutritional content has occurred. The producer knows the source of each element of the ready-to-eat dinner, how it was processed, including the recipe followed, and how it was preserved and packaged for the consumer. Not only does such a producer know what degradation to caloric and nutritional occurred, the producer can modify its processing and post-processing preservation to minimally affect nutritional content. The preparation of the nutritional substance for consumption can also degrade the nutritional content of nutritional substances. Finally, the consumer knows how she prepared the dinner, what condiments were added, and whether she did or did not enjoy it.

If there was a mechanism to share this information, the quality of the nutritional substances, including caloric and nutritional, organoleptic, and aesthetic value, could be preserved and improved. Consumers could be better informed about nutritional substances they select and consume, including the state, and changes in the state, ΔN, of the nutritional substance throughout its lifecycle from creation up to the moment of consumption. The efficiency and cost effectiveness of nutritional substances could also be improved. Feedback within the entire chain from creator to consumer could provide a closed-loop system that could improve quality (taste, appearance, and caloric and nutritional content), efficiency, value and profit. For example, in the milk supply chain, at least 10% of the milk produced is wasted due to safety margins included in product expiration dates. The use of more accurate tracking information, measured quality (including nutritional content) information, and historical environmental information could substantially reduce such waste. An interactive system and data base including dynamic nutritional substance labeling for collecting, preserving, measuring and/or tracking information about a nutritional substance in the nutritional substance supply system, would allow needed accountability. There would be nothing to hide. Unfortunately, today there is no such system or dynamic nutritional substance labeling.

As consumers are demanding more information about what they consume, they are asking for products that have higher nutritional content and more closely match good nutritional requirements, and would like nutritional products to actually meet their specific nutritional requirements. While grocery stores, restaurants, and all those who process and sell food and beverages may obtain some information from current nutritional substance tracking systems, such as existing non-dynamic nutritional substance labeling, these current systems can provide only limited information.

Current packaging materials for nutritional substances include plastics, paper, cardboard, glass, and synthetic materials. Generally, the packaging material is chosen by the producer to best preserve the quality of the nutritional substance until used by the customer. In some cases, the packaging may include some information regarding type of nutritional substance, identity of the producer, and the country of origin. Such packaging generally does not transmit source information of the nutritional substance, such as creation information, current or historic information as to the external conditions of the packaged nutritional substance, or current or historic information as to the internal conditions of the packaged nutritional substance.

Nutritional substance collectors and/or producers, such as growers (plants), ranchers (animals) or synthesizer (synthetic compounds), routinely create and collect information about their products, however, that information is generally not accessible by their customers. Even if such producers wished to provide such information to their customers, there is no current method of labeling, encoding or identifying each particular product to provide such information (even though all plants, animals and in general, nutritional substances have a natural fingerprint). While there are limited methods and systems available, they are excessively costly, time consuming, and do not trace, or provide access to, the nutritional substance organoleptic and/or nutritional state across the product's lifecycle. Current labels for such products include package labels, sticker labels and food color ink labels. These labels generally are applied to all similar products and cannot identify each particular product, only variety of products, such as apple banana, but not a particular banana.

An important issue in the creation, preservation, transformation, conditioning, and consumption of nutritional substances are the changes in nutritional, organoleptic, or aesthetic values, ΔN, that occur in nutritional substances due to a variety of internal and external factors. Because nutritional substances are composed of biological, organic, and/or chemical compounds, they are generally subject to degradation. This degradation generally reduces the nutritional, organoleptic, and/or aesthetic values of nutritional substances. While not always true, nutritional substances are best consumed at their point of creation. However, being able to consume nutritional substances at the farm, at the slaughterhouse, at the fishery, or at the food processing plant is at least inconvenient, if not impossible. Currently, the food and beverage industry attempts to minimize the loss of nutritional value (often through the use of additives or preservatives), and/or attempts to hide this loss of nutritional value from consumers.

Overall, the examples herein of some prior or related systems and their associated limitations are intended to be illustrative and not exclusive. Other limitations of existing or prior systems will become apparent to those of skill in the art upon reading the following Detailed Description.

### OBJECTS OF THE INVENTION

It is an object of the present invention to preserve a nutritional substance such that its source information and historical preservation information, including external influences on the nutritional substance which may have caused changes in nutritional, organoleptic, and/or aesthetic values of the nutritional substance, herein collectively and individually referred to as ΔN, and information regarding such ΔNs or a resulting nutritional, organoleptic, and/or aesthetic value, are available to users and/or consumers of the nutritional substance, as well as all entities of the nutritional substance supply system, including those who transform and condition nutritional substances.

A further object of the present invention is to provide packaging which dynamically interacts with the nutritional substance to maintain and/or improve and/or minimize degradation of the nutritional substance being preserved, in order to maintain, improve, or minimize degradation of a nutritional, organoleptic, and/or aesthetic value, or otherwise favorably influence a ΔN related to the nutritional substance.

It is an object of the present invention to preserve the nutritional substance such that its source information and/or historical preservation information, including external influences on the nutritional substance which may have caused changes in nutritional, organoleptic, and/or aesthetic values of the nutritional substance, herein collectively and individually referred to as ΔN, and information regarding such ΔNs or a resulting nutritional, organoleptic, and/or aesthetic value, are available to entities outside of the nutritional substance supply system.

In an object of the present invention, the packaging or label of a nutritional substance tracks creation and historical information of nutritional substance, including ΔN information as well as current information about the state of a nutritional, organoleptic, and/or aesthetic value of the nutritional substance.

A further object of the present invention is to provide packaging which dynamically interacts with the nutritional substance to maintain and/or improve and/or minimize degradation of the nutritional substance being preserved, in order to maintain, improve, or minimize degradation of a nutritional, organoleptic, and/or aesthetic value, or otherwise favorably influence a ΔN related to the nutritional substance, and transmits information regarding such dynamic interaction with the nutritional substance.

It is an object of the present invention to preserve the nutritional substance such that its source information and/or historical preservation information, including external influences on the nutritional substance which may have caused changes in nutritional, organoleptic, and/or aesthetic values of the nutritional substance, herein collectively and individually referred to as ΔN, and information regarding such ΔNs or a resulting nutritional, organoleptic, and/or aesthetic value, are available by reference to a unique identifier provided with the nutritional substance.

It is an object of the present invention to minimize and/or track ΔN of a nutritional substance, and collect, store, and transmit the ΔN information regarding the nutritional substance, and reference the ΔN information to a unique identifier associated with the nutritional substance.

### SUMMARY OF THE INVENTION

In one embodiment of the present invention, the preservation system, also referred to herein as packaging, for a nutritional substance allows for the tracking of source information, information as to the history of the nutritional substance from the point it was packaged and/or current information on external influences on the packaged nutritional substance which may have caused changes in nutritional, organoleptic, and/or aesthetic values of the nutritional substance, herein collectively and individually referred to as ΔN. It is a further embodiment of the present invention that the current information on the external influences on the packaged nutritional substance is utilized to provide ΔN values or resulting nutritional, organoleptic, and/or aesthetic values to users and/or consumers of the nutritional substance as well as all entities of the nutritional substance supply system, including those who transform and condition nutritional substances.

In another embodiment of the present invention the packaging or labeling for the nutritional substance can provide information to any entity inside or outside of the nutritional substance supply system, but preferably the consumer, related to a ΔN value or resulting nutritional, organoleptic, and/or aesthetic value of the nutritional substance.

In a further embodiment of the present invention, the packaging of the nutritional substance can dynamically interact with the nutritional substance to maintain, improve, or minimize degradation of a nutritional, organoleptic, and/or aesthetic value, or otherwise favorably influence a ΔN related to the nutritional substance.

In one embodiment of the present invention, the packaging, for a nutritional substance allows for the tracking of source information, information as to the history of the nutritional substance from the point it was packaged and/or current information on external influences on the packaged nutritional substance which may have caused changes in nutritional, organoleptic, and/or aesthetic values of the nutritional substance, herein collectively and individually referred to as ΔN. It is a further embodiment of the present invention that the current information on the external influences on the packaged nutritional substance is utilized to provide ΔN values or resulting nutritional, organoleptic, and/or aesthetic values to entities outside of the nutritional substance supply system.

In another embodiment of the present invention the packaging or labeling for the nutritional substance references information related to a ΔN value or resulting nutritional, organoleptic, and/or aesthetic value of the nutritional substance by a unique identifier provided by the packaging or labeling.

In a further embodiment of the present invention, the packaging of the nutritional substance can dynamically interact with the nutritional substance to maintain, improve, or minimize degradation of a nutritional, organoleptic, and/or aesthetic value, or otherwise favorably influence a ΔN related to the nutritional substance and transmits information related to the interaction.

In one embodiment of the present invention, the packaging, for a nutritional substance allows for the tracking of source information, information as to the history of the nutritional substance from the point it was packaged and/or current information on external influences on the packaged nutritional substance which may have caused changes in nutritional, organoleptic, and/or aesthetic values of the nutritional substance, herein collectively and individually referred to as ΔN. It is a further embodiment of the present invention that the current information on the external influences on the packaged nutritional substance is referenced to a unique identifier provided with the packaging.

An embodiment of the present invention provides a system for the creation, collection, storage, transmission, and/or processing of information regarding dynamically labeled nutritional substances so as to improve, maintain, or minimize degradation of nutritional, organoleptic, and/or aesthetic value of the nutritional substances. Additionally, the present invention provides such information for use by the creators, preservers, transformers, conditioners, and consumers of nutritional substances. It is a preferred that this information is openly available and openly integrated at any point in time to all constituents in the nutritional substance supply system. It is preferred that dynamic labeling provided with the nutritional substances enables the integration and availability of the information and that this information becomes openly available and openly integrated as soon as it is created. The nutritional information creation, preservation, and transmission system of the present invention should allow the nutritional substance supply system to improve its ability to minimize degradation of nutritional, organoleptic and/or aesthetic value of the nutritional substance, and/or inform the consumer, creator, packager, transformer, or conditioner about such degradation, or ΔN. While the ultimate goal of the nutritional substance supply system is to minimize degradation of nutritional, organoleptic and/or aesthetic values, or as it relates to ΔN, minimize the negative magnitude of ΔN. However, an interim goal should be providing consumers with significant information regarding any change, particularly degradation, of nutritional, organoleptic and/or aesthetic values of nutritional substances, and/or component nutritional substances thereof, consumers select and consumer, the ΔN, such that desired information regarding specific residual nutritional, organoleptic, and/or aesthetic values can be ascertained using the ΔN. Entities within the nutritional substance supply system that provide such ΔN information regarding nutritional substances, particularly regarding degradation, will be able to differentiate their products from those who obscure and/or hide such information. Additionally, such entities should be able to charge a premium for products which either maintain their nutritional, organoleptic, and/or aesthetic value, or supply more complete information about changes in their nutritional, organoleptic, and/or aesthetic value, the ΔN.

Other advantages and features will become apparent from the following description and claims. It should be understood that the description and specific examples are intended for purposes of illustration only and not intended to limit the scope of the present disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 shows a schematic functional block diagram of a nutritional substance supply relating to the present invention;

FIG. 2 shows a graph representing a value of a nutritional substance which changes according to a change of condition for the nutritional substance;

FIG. 3 shows a schematic functional block diagram of the preservation module 300 according to the present invention;

FIG. 4 shows a schematic functional block diagram of the preservation module 300 according to an alternate embodiment of the present invention;

FIG. 5 shows a schematic functional block diagram of the preservation module 300 according to an alternate embodiment of the present invention;

FIG. 6 shows a schematic functional block diagram of the preservation module 300 according to an alternate embodiment of the present invention;

FIG. 7 shows a schematic functional block diagram of the preservation module 300 according to an alternate embodiment of the present invention;

FIG. 8 shows a schematic functional block diagram of the preservation module 300 according to an alternate embodiment of the present invention;

FIG. 9 shows a schematic functional block diagram of the preservation module 300 according to an alternate embodiment of the present invention;

FIG. 10 shows a schematic functional block diagram of the preservation module 300 according to an alternate embodiment of the present invention;

FIG. 11 shows a schematic functional block diagram of the preservation module 300 according to an alternate embodiment of the present invention;

FIG. 12 shows a schematic functional block diagram of the preservation module 300 according to an alternate embodiment of the present invention; and

FIG. 13 shows a schematic functional block diagram of the preservation module 300 according to an alternate embodiment of the present invention.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION OF THE INVENTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

The following discussion provides a brief, general description of a representative environment in which the invention can be implemented. The present invention enables a nutritional substance to interact and communicate with its preservation system in a dynamic manner through the natural changes ΔN it experiences, and further enables the preservation system to convey information associated with those changes to the consumer. As used herein, preservation systems may include, but are not limited to, any internal or external portion of a nutritional substance package, container, carton, bottle, carton, box, bag, vessel, cup, plate, wrapper, label, or any other apparatus used to preserve, store, transfer, present, or serve a nutritional substance.

An example of the present invention is provided of bottled wine interacting, or communicating, with a portion of its container. As the wine in the container ages it naturally experiences many changes ΔN, including changes in acidity, tannin content, gas emission, sugar content, alcohol content, and others. According to the present invention, a cork, a cap, a submerged coupon or indicator, or any part of the surface of the bottle can monitor one or more ΔN and convey to a consumer the ΔN, or a corresponding current state, of the wine at any moment the consumer wants to know, such as when he is deciding to purchase or open the container.

In another example, a milk carton containing milk could have a small area on its side with encapsulated gel in direct contact with the milk. As the milk ages, its bacteria count naturally increases, also resulting in a reduced ph. The bacteria will be able to penetrate the gel and the gel will gradually change color in response to the increasing bacteria content or concentration, indicating the increase in bacteria within the milk, and therefore a current state of the milk. For example, the gel may change from green, wherein green represents an acceptable bacteria level and associated shelf life, to yellow, wherein yellow represents a higher acceptable bacteria level and associated shorter shelf life, to red, wherein red represents the milk has an unacceptably high bacteria level and is not apt for drinking any more.

Alternatively, the gel may gradually change color in response to a reduction in pH, wherein changes in ph are surrogates for changes in bacteria levels. As the milk ages, its bacteria count naturally increases, reducing its pH. For example, the gel may change from green, wherein green represents a pH level corresponding to an acceptable bacteria level and associated shelf life, to yellow, wherein yellow represents a lower pH level and corresponding higher acceptable bacteria level and associated shorter shelf life, to red, wherein red represents a still lower pH and corresponding unacceptably high bacteria level and is not apt for drinking any more.

It is understood that nutritional substances, as used herein, includes, but is not limited to, synthetic compounds such as medicaments, supplements, and other substances intended for consumption or introduction into a consumer. The present invention may include embodiments wherein a portion of the nutritional substance interacting or communicating with its container is segregated from a portion of the nutritional substance to be consumed. This would be of particular benefit for packaged goods including synthetic compounds such as medicaments, in which case it would be desirable to segregate the portion of medicament interacting or communicating with the container from the portion of the medicament for consumption. In this case, the portion of the medicament interacting or communicating with the container would serve as a parallel sample of the medicament provided for consumption. This might be accomplished by providing a separate, permanently sealed cavity on or within the medicament container, its cover, its label, or any permanently sealed cavity structure known in the art, wherein the structure contains the portion of medicament intended to interact or communicate with the container. The permanently sealed cavity can interact with the portion of medicament communicating with it to convey desired ΔN information regarding the medicament. Such ΔN information may be associated with a degradation of the medicament, a residual value of the medicament, an expiration date of the medicament, or utilized in any other way to ensure the medicament's safety and efficacy when a consumer uses it.

Other examples of the present invention could include, but are not limited to, containers like jars, glasses, or cups that could detect when there is an unhealthy level of toxins, antibiotics, fungus, bacteria, pesticides, or other undesirable components in tap water intended for consumption, or if the coffee poured into a cup has caffeine or not. The principle at work is that of symbiosis, similar to that which occurs between a banana and its peel. The banana peel has a natural evolution from green to black that conveys the level of maturity of the banana. The peel reacts to the natural ΔN that occurs during the banana's maturation process, wherein the ΔNs may include changes in acidity, sugar content, and bacteria level. The ΔNs of the banana independently and collectively have an effect on the aesthetic values of the banana peel, which in turn conveys to the consumer when and how the banana may best be consumed. For example, a green peel indicates that the banana is not yet ripe and should not be eaten. Yellow indicates that it may be suitable for consumption, but will not be very sweet. Yellow with a few black spots indicates that it is suitable for consumption, and will be sweat. Mostly black indicates that it is suitable for use in baked goods or to be fried. Very black indicates that it is no longer suitable for consumption. In this same manner when the peel has been punctured or torn and the maturating process is accelerated as more oxygen than normal contacts the banana, the banana peel quickly turns black alerting the consumer. Therefore the consumer does not have to rely on a static expiration date to determine the banana's suitability for consumption.

Although not required, aspects of the invention may be described below in the general context of computer-executable instructions, such as routines executed by a general-purpose data processing device (e.g., a server computer or a personal computer). Those skilled in the relevant art will appreciate that the invention can be practiced with other communications, data processing, or computer system configurations, including: wireless devices, Internet appliances, hand-held devices (including personal digital assistants (PDAs)), wearable computers, all manner of cellular or mobile phones, multi-processor systems, microprocessor-based or programmable consumer electronics, set-top boxes, network PCs, mini-computers, mainframe computers, and the like. Indeed, the terms “controller,” “computer,” “server,” and the like are used interchangeably herein, and may refer to any of the above devices and systems.

While aspects of the invention, such as certain functions, are described as being performed exclusively on a single device, the invention can also be practiced in distributed environments where functions or modules are shared among disparate processing devices. The disparate processing devices are linked through a communications network, such as a Local Area Network (LAN), Wide Area Network (WAN), or the Internet. In a distributed computing environment, program modules may be located in both local and remote memory storage devices.

Aspects of the invention may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data related to the invention may be distributed over the Internet or over other networks (including wireless networks), on a propagated signal on a propagation medium (e.g., an electromagnetic wave(s), a sound wave, etc.) over a period of time. In some implementations, the data may be provided on any analog or digital network (packet switched, circuit switched, or other scheme).

In some instances, the interconnection between modules is the internet, allowing the modules (with, for example, WiFi capability) to access web content offered through various web servers. The network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), etc.

The modules in the systems can be understood to be integrated in some instances and in particular embodiments, only particular modules may be interconnected.

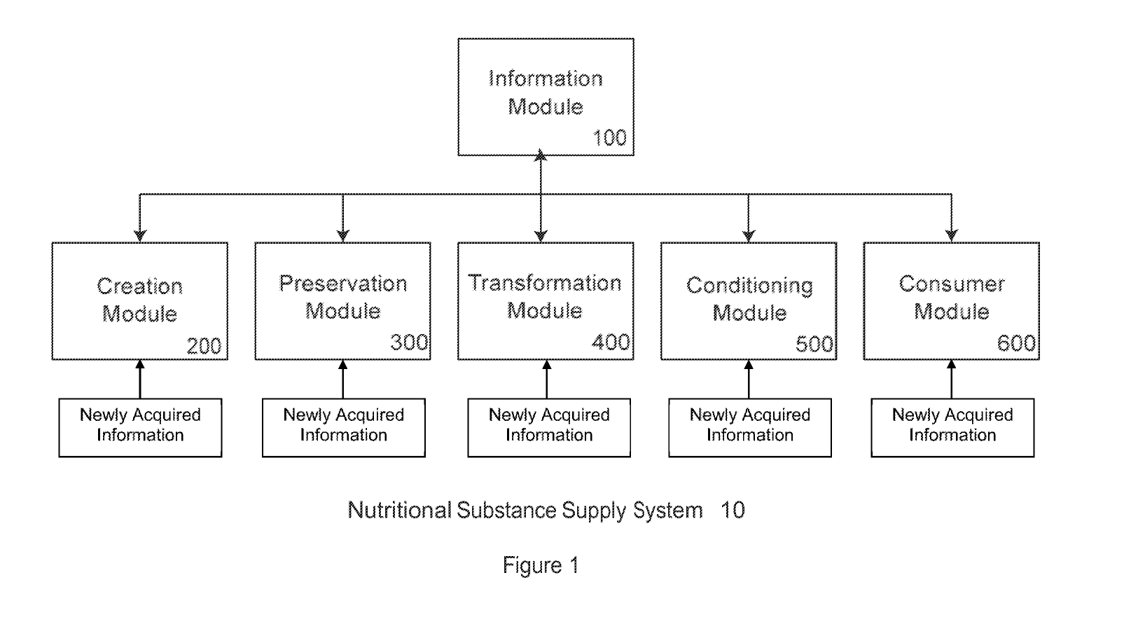

FIG. 1 shows the components of a nutritional substance industry 10. It should be understood that this could be the food and beverage ecosystem for human consumption, but could also be the feed industry for animal consumption, such as the pet food industry. A goal of the present invention for nutritional substance industry 10 is to create, preserve, transform and trace change in nutritional, organoleptic and/or aesthetic values of nutritional substances, collectively and individually also referred to herein as ΔN, through their creation, preservation, transformation, conditioning and consumption. While the nutritional substance industry 10 can be composed of many companies or businesses, it can also be integrated into combinations of business serving many roles, or can be one business or even individual. Since ΔN is a measure of the change in a value of a nutritional substance, knowledge of a prior value (or state) of a nutritional substance and the ΔN value will provide knowledge of the changed value (or state) of a nutritional substance, and can further provide the ability to estimate a change in value (or state).

Module 200 is the creation module. This can be a system, organization, or individual which creates and/or originates nutritional substances. Examples of this module include a farm which grows produce; a ranch which raises beef; an aquaculture farm for growing shrimp; a factory that synthesizes nutritional compounds; a collector of wild truffles; or a deep sea crab trawler.

Preservation module 300 is a preservation system for preserving and protecting the nutritional substances created by creation module 200. Once the nutritional substance has been created, generally, it will need to be packaged in some manner for its transition to other modules in the nutritional substances industry 10. While preservation module 300 is shown in a particular position in the nutritional substance industry 10, following the creation module 200, it should be understood that the preservation module 300 actually can be placed anywhere nutritional substances need to be preserved during their transition from creation to consumption.

Transformation module 400 is a nutritional substance processing system, such as a manufacturer who processes raw materials such as grains into breakfast cereals. Transformation module 400 could also be a ready-to-eat dinner manufacturer who receives the components, or ingredients, also referred to herein as component nutritional substances, for a ready-to-eat dinner from preservation module 300 and prepares them into a frozen dinner. While transformation module 400 is depicted as one module, it will be understood that nutritional substances may be transformed by a number of transformation modules 400 on their path to consumption.

Conditioning module 500 is a consumer preparation system for preparing the nutritional substance immediately before consumption by the consumer. Conditioning module 500 can be a microwave oven, a blender, a toaster, a convection oven, a cook, etc. It can also be systems used by commercial establishments to prepare nutritional substance for consumers such as a restaurant, an espresso maker, pizza oven, and other devices located at businesses which provide nutritional substances to consumers. Such nutritional substances could be for consumption at the business or for the consumer to take out from the business. Conditioning module 500 can also be a combination of any of these devices used to prepare nutritional substances for consumption by consumers.

Consumer module 600 collects information from the living entity which consumes the nutritional substance which has passed through the various modules from creation to consumption. The consumer can be a human being, but could also be an animal, such as pets, zoo animals and livestock, which are they themselves nutritional substances for other consumption chains. Consumers could also be plant life which consumes nutritional substances to grow.

Information module 100 receives and transmits information regarding dynamically labeled nutritional substances between each of the modules in the nutritional substance industry 10 including, the creation module 200, the preservation module 300, the transformation module 400, the conditioning module 500, and the consumer module 600. The nutritional substance information module 100 can be an interconnecting information transmission system which allows the transmission of information between various modules. It is preferred that the information module 100 collects, tracks, and organizes information regarding the dynamically-labeled nutritional substances from each stage of the production of the nutritional substances from creation to consumption and that the information regarding the dynamically-labeled nutritional substances is openly available and openly integrated at any point in time to all modules of the nutritional substance supply system, preferably as soon as it is created. The integration and availability of the information is enabled by dynamic labeling provided with the nutritional substances, which includes a unique nutritional substance identifier, also referred to herein as a dynamic information identifier. Information module 100 contains a database, also referred to herein as a dynamic nutritional value database, where the information regarding the dynamically labeled nutritional substance resides and can be referenced or located by the corresponding dynamic information identifier. Information module 100 can be connected to the other modules by a variety of communication systems, such as paper, computer networks, the Internet and telecommunication systems, such as wireless telecommunication systems.

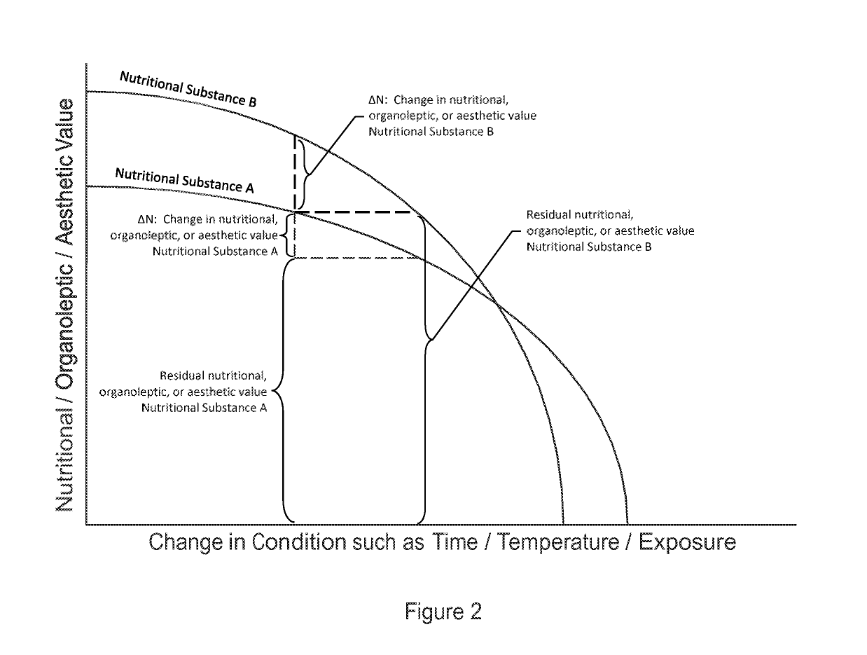

FIG. 2 is a graph showing the function of how a nutritional, organoleptic, or aesthetic value of a nutritional substance varies over the change in a condition of the nutritional substance. Plotted on the vertical axis of this graph can be either the nutritional value, organoleptic value, or even the aesthetic value of a nutritional substance. Plotted on the horizontal axis can be the change in condition of the nutritional substance, ΔN, over a variable such as time, temperature, location, and/or exposure to environmental conditions (this is indicated as “ΔN: Change in nutritional, organoleptic, or aesthetic value” in FIG. 2) Also shown in FIG. 2 is the residual nutritional, organoleptic, or aesthetic value of the nutritional substance (indicated by “Residual nutritional, organoleptic, or aesthetic value”). This exposure to environmental conditions can include: exposure to air, including the air pressure and partial pressures of oxygen, carbon dioxide, water, or ozone; airborne chemicals, pollutants, allergens, dust, smoke, carcinogens, radioactive isotopes, or combustion byproducts; exposure to moisture; exposure to energy such as mechanical impact, mechanical vibration, irradiation, heat, or sunlight; or exposure to materials such as packaging. The function plotted as nutritional substance A could show a ΔN for milk, such as the degradation of a nutritional value of milk over time. Any point on this curve can be compared to another point to measure and/or describe the change in nutritional value, or the ΔN of nutritional substance A. The plot of the degradation in the same nutritional value of nutritional substance B, also milk, describes the change in nutritional value, or the ΔN of nutritional substance B, a nutritional substance which starts out with a higher nutritional value than nutritional substance A, but degrades over time more quickly than nutritional substance A.

In this example, where nutritional substance A and nutritional substance B are milk, this ΔN information regarding the nutritional substance degradation profile of each milk could be used by the consumer in the selection and/or consumption of the milk if nutritional substance A and nutritional substance B are provided with dynamic labeling, which would include a dynamic information identifier for each nutritional substance. Using the dynamic information identifier obtained from the dynamic labeling provided with each nutritional substance, the consumer could retrieve desired ΔN information, such as the nutritional substance degradation profile referenced to each of the milks, from a dynamic nutritional value database. If the consumer has this information at time zero when selecting a milk product for purchase, the consumer could consider when the consumer plans to consume the milk, whether that is on one occasion or multiple occasions. For example, if the consumer planned to consume the milk prior to the point when the curve represented by nutritional substance B crosses the curve represented by nutritional substance A, then the consumer should choose the milk represented by nutritional substance B because it has a higher nutritional value until it crosses the curve represented by nutritional substance A. However, if the consumer expects to consume at least some of the milk at a point in time after the time when the curve represented by nutritional substance B crosses the curve represented by nutritional substance A, then the consumer might choose to select the milk represented by the nutritional substance A, even though milk represented by nutritional substance A has a lower nutritional value than the milk represented by nutritional substance B at an earlier time. This change to a desired nutritional value in a nutritional substance, ΔN, over a change in a condition of the nutritional substance described in FIG. 2 can be measured and controlled throughout nutritional substance supply system 10 in FIG. 1. This example demonstrates how dynamically generated information regarding a ΔN of a dynamically labeled nutritional substance, in this case a change in nutritional value of milk, can be used to understand a rate at which that nutritional value changes or degrades; when that nutritional value expires; and a residual nutritional value of the nutritional substance over a change in a condition of the nutritional substance, in this example a change in time. This ΔN information could further be used to determine a best consumption date for nutritional substance A and B, which could be different from each other depending upon the dynamically generated information generated for each.

There is also the ΔN as two or more nutritional substances combine. For example, when lemon is added to guacamole it keeps the avocado in the guacamole from turning black. The function plotted as nutritional substance A could show a ΔN for guacamole made by a first transformer, such as the degradation of an aesthetic value of guacamole over time, in this case a degradation of its green color. Any point on this curve can be compared to another point to measure and/or describe the change in aesthetic value, or the ΔN of nutritional substance A. The plot of the degradation in the same aesthetic value of nutritional substance B, a guacamole made by a second transformer, describes the change in the same aesthetic value, or the ΔN, of nutritional substance B. Nutritional substance B starts out with a higher aesthetic value than nutritional substance A, but degrades over time more quickly than nutritional substance A, for instance because the transformer of nutritional substance B adds less lemon juice to their guacamole in order not to distract from the flavor of the avocado. The information available is related to the interaction of the avocado and lemon juice in the respective manufacturer's guacamole, and can enable the consumer to make decisions related to the aesthetic value of the guacamole at a given point in time if nutritional substance A and nutritional substance B are provided with dynamic labeling, which would include a dynamic information identifier for each nutritional substance. Using the dynamic information identifier obtained from the dynamic labeling provided with each nutritional substance, the consumer could retrieve desired ΔN information, such as the aesthetic degradation profile referenced to each guacamole, from a dynamic nutritional value database. For example, if the consumer is purchasing the guacamole to consume at a time before the two curves intersect, and the decision is based on superior aesthetic value, the consumer will choose nutritional substance B. If the consumer is purchasing the guacamole to consume after the time the two curves intersect, and the decision is based on superior aesthetic value, the consumer will choose nutritional substance A, even though it has lower aesthetic value at the time of purchase.

In another example, the lemon has been added to sliced apples to keep the sliced apples from turning black. The function plotted as nutritional substance A could show a ΔN for sliced apples transformed by a first transformer, such as the degradation of the aesthetic value of the sliced apples over time, in this case a degradation of its pale color. Any point on this curve can be compared to another point to measure and/or describe the change in aesthetic value, or the ΔN of nutritional substance A. The plot of the degradation in the same aesthetic value of nutritional substance B, sliced apples made by a slightly different process by a second transformer, describes the same change in the aesthetic value, or the ΔN, of nutritional substance B. Nutritional substance B starts out with a higher aesthetic value than nutritional substance A, but degrades over time more quickly than nutritional substance A, for instance because the manufacturer of nutritional substance B adds less lemon juice to their sliced apples in order not to distract from the flavor of the apples. The information available is related to the interaction of the apples and lemon juice in the respective transformer's sliced apples, and can enable the consumer to make decisions related to the aesthetic value of the sliced apples at a given point in time if nutritional substance A and nutritional substance B are provided with dynamic labeling, which would include a dynamic information identifier for each nutritional substance. Using the dynamic information identifier obtained from the dynamic labeling provided with each nutritional substance, the consumer could retrieve desired ΔN information, such as the aesthetic degradation profile referenced to the sliced apples of each transformer, from a dynamic nutritional value database. For example, if the consumer is purchasing the sliced apples to consume before the time the two curves intersect, and the decision is based on superior aesthetic value, the consumer will choose nutritional substance B. If the consumer is purchasing the sliced apples to consume after the time the two curves intersect, and the decision is based on superior aesthetic value, the consumer will choose nutritional substance A, even though it has lower aesthetic value at the time of purchase.

In FIG. 1, Creation module 200 can dynamically encode nutritional substances, as part of the nutritional substance dynamic labeling, to enable the tracking of changes in nutritional, organoleptic, and/or aesthetic value of the nutritional substance, or ΔN. This dynamic encoding, also referred to herein as a dynamic information identifier, can replace and/or complement existing nutritional substance marking systems such as barcodes, labels, and/or ink markings. This dynamic encoding, or dynamic information identifier, can be used to make nutritional substance information from creation module 200 available to information module 100 for use by preservation module 300, transformation module 400, conditioning module 500, and/or consumption module 600, which includes the ultimate consumer of the nutritional substance. A key resource also available through module 100 is recipe information regarding meals that may utilize the nutritional substances as components. The ΔN information combined with recipe information from module 100 will not only be of great benefit to the consumer in understanding and accomplishing the nutritional, organoleptic, and aesthetic values desired, it will even help dispel misunderstandings that consumers may have about particular nutritional, organoleptic, and aesthetic values of nutritional substances or the combination or nutritional substances. One method of providing dynamically labeled nutritional substances with a dynamic information identifier by creation module 200, or any other module in nutritional supply system 10, could include an electronic tagging system, such as the tagging system manufactured by Kovio of San Jose, Calif., USA. Such thin film chips can be used not only for tracking nutritional substances, but can include components to measure attributes of nutritional substances, and record and transmit such information. Such information may be readable by a reader including a satellite-based system. Such a satellite-based nutritional substance information tracking system could comprise a network of satellites with coverage of some or all the surface of the earth, so as to allow the dynamic nutritional value database of information module 100 real time, or near real time updates about a ΔN of a particular nutritional substance. In turn, this information is openly available and openly integrated at any point in time to all constituents in the nutritional substance supply system. It is also preferred that this information becomes openly available and openly integrated as soon as it becomes available.

Preservation module 300 includes packers and shippers of nutritional substances. The tracking of changes in nutritional, organoleptic, and/or aesthetic values, or a ΔN, during the preservation period within preservation module 300 allows for dynamic expiration dates for nutritional substances. For example, expiration dates for dairy products are currently based generally only on time using assumptions regarding minimal conditions at which dairy products are maintained. This extrapolated expiration date is based on a worst-case scenario for when the product becomes unsafe to consume during the preservation period. In reality, the degradation of dairy products may be significantly less than this worst-case. If preservation module 300 could measure or derive the actual degradation information such as ΔN, an actual expiration date, referred to herein as a dynamic expiration date, can be determined dynamically, and could be significantly later in time than an extrapolated expiration date. This would allow the nutritional substance supply system to dispose of fewer products due to expiration dates. This ability to dynamically generate expiration dates for nutritional substances is of particular significance when nutritional substances contain few or no preservatives. Such products are highly valued throughout nutritional substance supply system 10, including consumers who are willing to pay a premium for nutritional substances with few or no preservatives. Consumers of nutritional substances provided with dynamic labeling comprising dynamic information identifiers according to the present invention could readily access information regarding dynamic expiration dates for the nutritional substances.

It should be noted that a dynamic expiration date need not be indicated numerically (i.e., as a numerical date) but could be indicated symbolically as by the use of colors—such as green, yellow and red employed on semaphores—or other designations. In those instances, the dynamic expiration date would not be interpreted literally but, rather, as a dynamically-determined advisory date. In practice a dynamic expiration date will be provided for at least one component of a single or multi-component nutritional substance. For multi-component nutritional substances, the dynamic expiration date could be interpreted as a “best” date for consumption for particular components. Consumers of nutritional substances provided with dynamic labeling comprising dynamic information identifiers according to the present invention could readily access this type of information regarding dynamic expiration dates for the nutritional substances.

By law, in many localities, food processors such as those in transformation module 400 are required to provide nutritional substance information regarding their products. Often, this information takes the form of a nutritional table applied to the packaging of the nutritional substance. Currently, the information in this nutritional table is based on averages or minimums for their typical product. Using the nutritional substance information from information module 100 provided by creation module 200, preservation module 300, and/or information from the transformation of the nutritional substance by transformation module 400, the food processor could include a dynamically generated nutritional value table, also referred to herein as a dynamic nutritional value table, for the actual dynamically-labeled nutritional substance being supplied. The information in such a dynamic nutritional value table could be used by conditioning module 500 in the preparation of the dynamically-labeled nutritional substance, and/or used by consumption module 600, so as to allow the ultimate consumer the ability to select the most desirable dynamically-labeled nutritional substance which meets their needs, and/or to track information regarding dynamically-labeled nutritional substances consumed.

Information about changes in nutritional, organoleptic, and/or aesthetic values of nutritional substances, or ΔN, is particularly useful in the conditioning module 500 of the present invention, as it allows knowing, or estimating, the pre-conditioning state of the nutritional, organoleptic, and/or aesthetic values of the dynamically labeled nutritional substance, and allows for estimation of a ΔN associated with proposed conditioning parameters. The conditioning module 500 can therefore create conditioning parameters, such as by modifying existing or baseline conditioning parameters, which can exist as recipes and conditioning protocols available through the information module 100 or locally available through the conditioning module 500, to deliver desired nutritional, organoleptic, and/or aesthetic values after conditioning. The pre-conditioning state of the nutritional, organoleptic, and/or aesthetic value of a nutritional substance is not tracked or provided to the consumer by existing conditioners, nor is the ΔN expected from a proposed conditioning tracked or provided to the consumer either before or after conditioning. However, using information provided by information module 100 from creation module 200, preservation module 300, transformation module 400, and/or information measured or generated by conditioning module 500 and/or consumer information from the consumer module 600, conditioning module 500 could provide the consumer with the actual, and/or estimated change in nutritional, organoleptic, and/or aesthetic values of a dynamically-labeled nutritional substance, or ΔN. Such information regarding the change to nutritional, organoleptic and/or aesthetic value of the dynamically-labeled nutritional substance, or ΔN, could be provided not only to the consumer, but could also be provided to information module 100 for use by creation module 200, preservation module 300, transformation module 400, so as to track, and possibly improve nutritional substances throughout the entire nutritional substance supply system 10.

The information regarding nutritional substances provided by information module 100 to consumption module 600 can replace or complement existing information sources such as recipe books, food databases like www.epicurious.com, and Epicurious apps. Through the use of specific information regarding a dynamically-labeled nutritional substance from information module 100, consumers can use consumption module 600 to select nutritional substances according to nutritional, organoleptic, and/or aesthetic values. This will further allow consumers to make informed decisions regarding nutritional substance additives, preservatives, genetic modifications, origins, traceability, and other nutritional substance attributes that may also be tracked through the information module 100. This information can be provided by consumption module 600 through personal computers, laptop computers, tablet computers, and/or smartphones. Software running on these devices can include dedicated computer programs, modules within general programs, and/or smartphone apps. An example of such a smartphone app regarding nutritional substances is the iOS ShopNoGMO from the Institute for Responsible Technology. This iPhone app allows consumers access to information regarding non-genetically modified organisms they may select. Additionally, consumption module 600 may provide information for the consumer to operate conditioning module 500 in such a manner as to optimize nutritional, organoleptic, and/or aesthetic values of a dynamically-labeled nutritional substance and/or component nutritional substances thereof according to the consumer's needs or preference, and/or minimize degradation of, preserve, or improve nutritional, organoleptic, and/or aesthetic value of a dynamically-labeled nutritional substance and/or component nutritional substances thereof.

Through the use of nutritional substance information available from information module 100 nutritional substance supply system 10 can track nutritional, organoleptic, and/or aesthetic value of dynamically-labeled nutritional substances. Using this information, dynamically-labeled nutritional substances travelling through nutritional substance supply system 10 can be dynamically valued and priced according to nutritional, organoleptic, and/or aesthetic values. For example, nutritional substances with longer dynamic expiration dates (longer shelf life) may be more highly valued than nutritional substances with shorter expiration dates. Additionally, nutritional substances with higher nutritional, organoleptic, and/or aesthetic values may be more highly valued, not just by the consumer, but also by each entity within nutritional substance supply system 10. This is because each entity will want to start with a nutritional substance with higher nutritional, organoleptic, and/or aesthetic value before it performs its function and passes the nutritional substance along to the next entity. Therefore, both the starting nutritional, organoleptic, and/or aesthetic value and the ΔN associated with those values are important factors in determining or estimating an actual, or residual, nutritional, organoleptic, and/or aesthetic value of a nutritional substance, and accordingly are important factors in establishing dynamically valued and priced nutritional substances.

During the period of implementation of the present inventions, there will be nutritional substances being marketed including those benefiting from dynamic labeling and the tracking of dynamic nutritional information such as ΔN, also referred to herein as information-enabled nutritional substances, and nutritional substances which do not benefit from dynamic labeling or the tracking of dynamic nutritional information such as ΔN, which are not information enabled and are referred to herein as dumb nutritional substances. Information-enabled nutritional substances would be available in virtual internet marketplaces, as well as traditional marketplaces. Because of information provided by information-enabled nutritional substances, entities within the nutritional substance supply system 10, including consumers, would be able to review and select information-enabled nutritional substances for purchase. It should be expected that, initially, the information-enabled nutritional substances would enjoy a higher market value and price than dumb nutritional substances. However, as information-enabled nutritional substances become more the norm, the cost savings from less waste due to degradation of information-enabled nutritional substances could lead to their price actually becoming less than dumb nutritional substances. Ultimately, an information system will evolve wherein information module 100 has the ability for creating traffic and signing on the address of users to not only facilitate the rapid adoption and utilization of better nutritional substance information according to the present invention, but also be a key source of business and revenue growth.

For example, the producer of a ready-to-eat dinner would prefer to use corn of a high nutritional, organoleptic, and/or aesthetic value in the production of its product, the ready-to-eat dinner, so as to produce a premium product of high nutritional, organoleptic, and/or aesthetic value. Depending upon the levels of the nutritional, organoleptic, and/or aesthetic values, the ready-to-eat dinner producer may be able to charge a premium price and/or differentiate its product from that of other producers. When selecting the corn to be used in the ready-to-eat dinner, the producer will seek corn of high nutritional, organoleptic, and/or aesthetic value from preservation module 300 that meets its requirements for nutritional, organoleptic, and/or aesthetic value. The packager/shipper of preservation module 300 would also be able to charge a premium for corn which has high nutritional, organoleptic, and/or aesthetic values. And finally, the packager/shipper of preservation module 300 will select corn of high nutritional, organoleptic, and/or aesthetic value from the grower of creation module 200, who will also be able to charge a premium for corn of high nutritional, organoleptic, and/or aesthetic values.

The change to nutritional, organoleptic, and/or aesthetic value for an information-enabled nutritional substance, or ΔN, tracked through nutritional substance supply system 10 through nutritional substance information from information module 100 can be preferably determined from measured information. However, some or all such nutritional substance ΔN information may be derived through measurements of environmental conditions of the nutritional substance as it traveled through nutritional substance supply system 10. Additionally, some or all of the information-enabled nutritional substance ΔN information can be derived from ΔN data of other information-enabled nutritional substances which have traveled through nutritional substance supply system 10. Information-enabled nutritional substance ΔN information can also be derived from laboratory experiments performed on other nutritional substances, which may approximate conditions and/or processes to which the actual information-enabled nutritional substance has been exposed. Further, consumer feedback and updates regarding observed or measured changes in the nutritional, organoleptic, and/or aesthetic value of information-enabled nutritional substances can play a role in updating ΔN information. Also, a creator, preserver, transformer, or conditioner may revise ΔN information, or information regarding other attributes of information-enabled nutritional substances they have previously created or processed, based upon newly acquired information affecting the ΔN or the other attributes.

For example, laboratory experiments can be performed on bananas to determine effect on or change in nutritional, organoleptic, and/or aesthetic value, or ΔN, for a variety of environmental conditions bananas may be exposed to during packaging and shipment in preservation module 300. Using this experimental data, tables and/or algorithms could be developed which would predict the level of change of nutritional, organoleptic, and/or aesthetic values, or ΔN, for a particular information-enabled banana based upon information collected regarding the environmental conditions to which the information-enabled banana was exposed during its time in preservation module 300. While the ultimate goal for nutritional substance supply system 10 would be the actual measurement of nutritional, organoleptic, and/or aesthetic values to determine ΔN, use of derived nutritional, organoleptic, and/or aesthetic values from experimental data to determine ΔN would allow improved logistics planning because it provides the ability to prospectively estimate changes to nutritional, organoleptic, and/or aesthetic values, or ΔN, and because it allows more accurate tracking of changes to nutritional, organoleptic, and/or aesthetic values, or ΔN, while technology and systems are put in place to allow actual measurement.

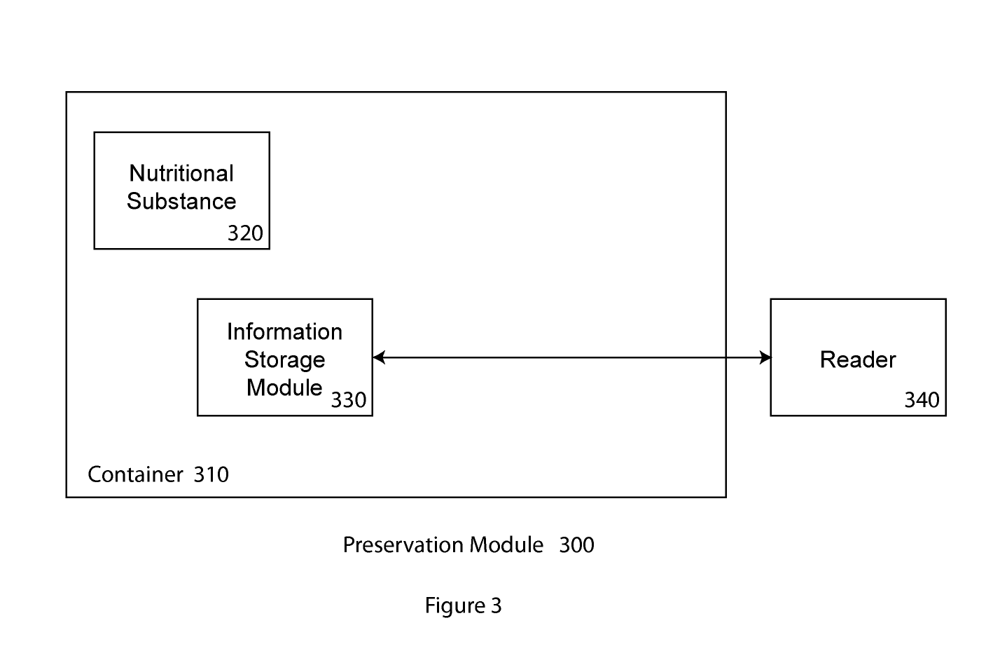

FIG. 3 shows an embodiment of the preservation module of the present invention. Preservation system 300 includes a container 310 which contains nutritional substance 320. Also included in container 310 is information storage module 330 which can be connected to an external reader 340. In this embodiment, information storage module 330 contains information regarding the nutritional substance 320. This information can include creation information from the creation of the nutritional substance 320. However, information in the information storage module 320 might include identification information, information regarding prior transformation of the nutritional substance 320, information related to ΔN, and other historic information. A shipper, or user, of container 310 can operatively connect to information storage module 330 using reader 340 to retrieve information stored therein.

In an alternate embodiment reader 340 can also write to information storage module 330. In this embodiment, information regarding the container and/or nutritional substance 320 can be modified or added to information storage module 330 by the user or shipper.

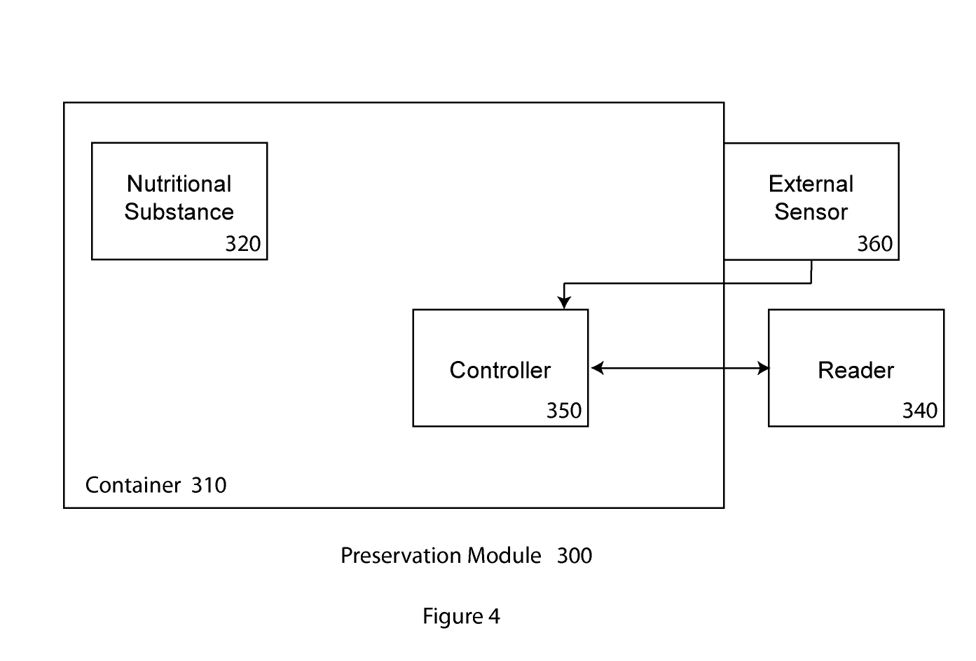

FIG. 4 shows another embodiment of preservation system 300 wherein container 310 contains nutritional substance 320 as well as controller 350. Controller 350 is connected to external sensor 360 located either inside, on the surface of, or external to container 310 such that external sensor 360 can obtain information regarding the environment external to container 310. Controller 350 and exterior sensor 360 can take the form of electronic components such as a micro-controller and an electronic sensor. However, the controller-sensor combination may also be chemical or organic materials which perform the same function, such as a liquid crystal sensor/display.

When the shipper or user of container 310 desires information from external sensor 360 the shipper or user can use reader 340 to query the controller 350 as to the state of external sensor 360. In the electronic component embodiment, reader 340 could be a user interface device such as a computer which can be electronically connected to controller 350. In the liquid crystal sensor/display, the ready could be a human looking at the display.

In one embodiment, reader 340 can be directly connected to external sensor 360 to obtain the information from external sensor 360 without need of a controller 350. In another embodiment, external sensor 360 provides information to controller 350 which is presented as a visual display to the shipper or user. Finally, external sensor 360 could provide information directly to the user or shipper by visual means such as a temperature sensitive liquid crystal thermometer.

In an additional embodiment, controller 350 can modify the operation of container 310 so as modify the preservation capabilities of container 310, so as to favorably influence a ΔN of the nutritional substance. For example, if the exterior environment of container 310 would adversely affect the nutritional substance 320, container 310 could adjust the internal environment of container 310 to better preserve the nutritional substance. If nutritional substance needs to be kept within a certain temperature range to preserve its organoleptic and/or nutritional properties, and the external sensor 360 provide exterior temperature information to controller 350, controller 350 could modify container 310 so as to maintain nutritional substance 320 within the required temperature range.

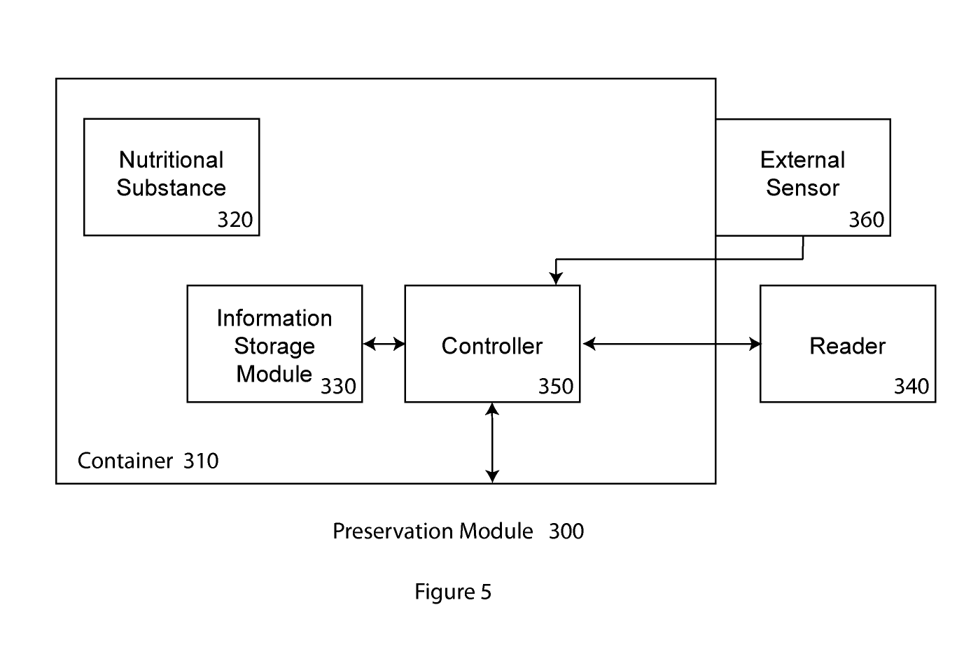

In FIG. 5, preservation system 300 includes container 310 which contains nutritional substance 320, controller 350, and information storage module 330. External sensor 360 is positioned such that it can provide information on the exterior environment to container 310. Information from the external sensor and information storage module can be retrieved by connecting reader 340 to container 310.

In this embodiment, information regarding the external environment sensed by external sensor 360 and provided to controller 350 can be stored in information storage module 330. This storage of external environment can be used to record a history the external environment container 310 has been subjected to. This would allow the shipper or user of container 310 to understand the external environment the container has been subjected to during the time it has preserved the nutritional substance. Such information can be used to determine any number of ΔN values for the nutritional substance and if the nutritional substance is no longer safe for consumption or has been degraded such that the nutritional substance is no longer in an optimal state. Additionally, the user of the nutritional substance could modify its transformation, conditioning, or consumption according to any changes, or ΔNs that may have occurred because of the external conditions of the container.

Additionally, in this embodiment, information storage module 340 could contain other information regarding the nutritional substance, including creation information, identification information, and/or prior transformation information.

In an additional embodiment, controller 350 can modify the operation of container 310 so as modify the preservation capabilities of container 310, so as to favorably influence a ΔN of the nutritional substance. For example, if the exterior environment of container 310 would adversely affect the nutritional substance 320, container 310 could adjust the internal environment of container 310 to better preserve the nutritional substance. Controller 350 can analyze the historic information from external sensor 360, stored in information storage module 330 to determine any long-term exterior conditions environmental If nutritional substance needs to be kept within a certain temperature range to preserve its organoleptic and/or nutritional properties, and the external sensor 360 provide exterior temperature information to controller 350, controller 350 could modify container 310 so as to maintain nutritional substance 320 within the required temperature range.

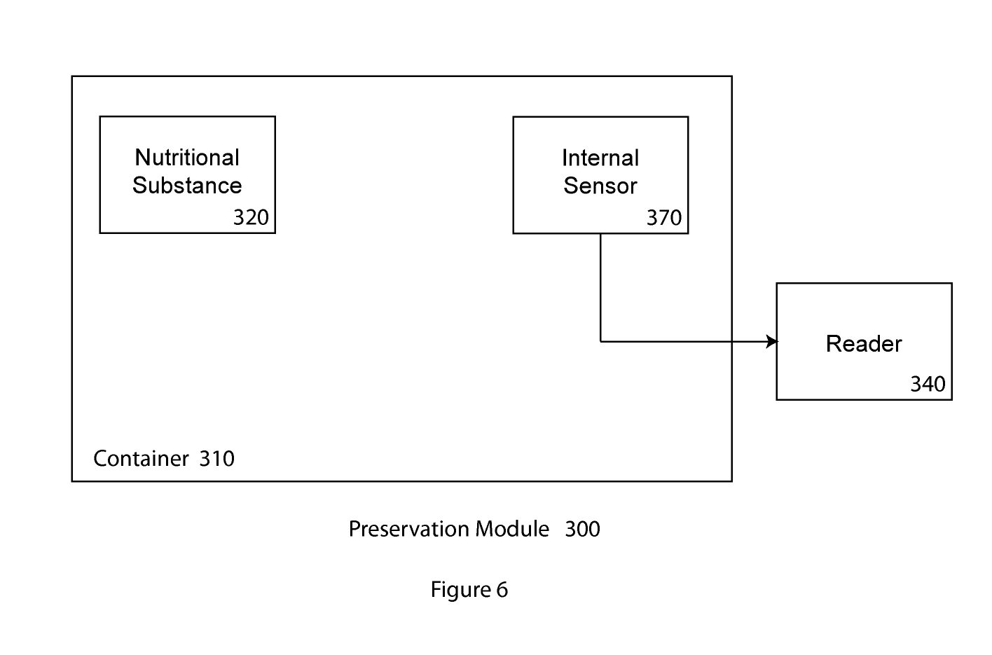

FIG. 6 shows embodiment of preservation system 300 wherein container 310 contains nutritional substance 320 as well as internal sensor 370 located either inside, or on the surface of, container 310, such that internal sensor 370 can obtain information regarding the environment internal to container 310. Internal sensor 370 can be connected to reader 340 to obtain the interior conditions of container 310. Internal sensor 360 and reader 340 can take the form of electronic components such as an electronic sensor and electronic display. However, the controller-sensor combination may also be chemical or organic materials which perform the same function, such as a liquid crystal sensor/display.

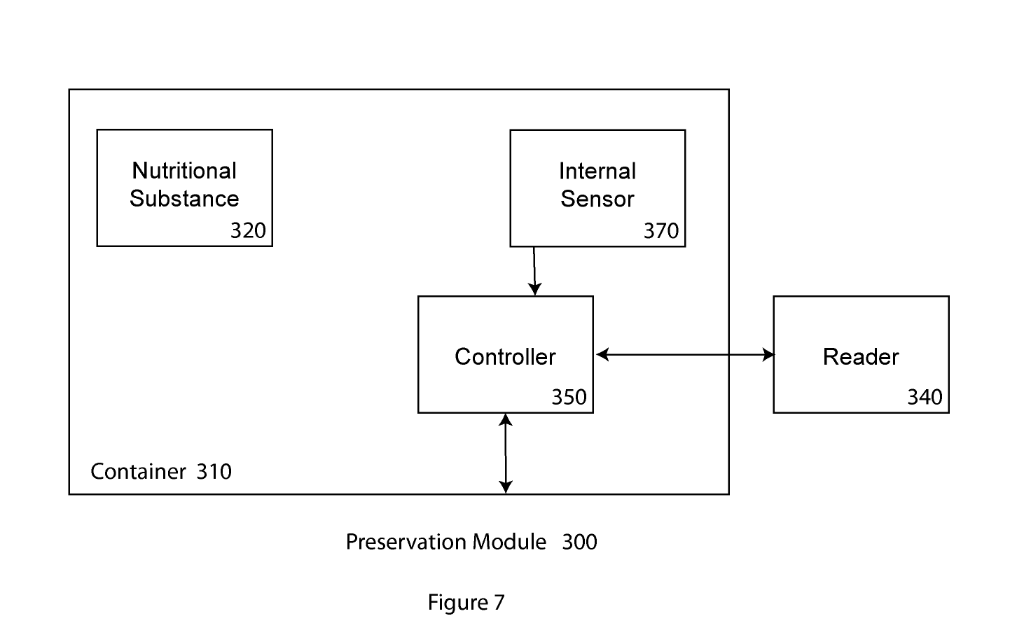

FIG. 7 shows embodiment of preservation system 300 wherein container 310 contains nutritional substance 320 as well as controller 350. Controller 350 is connected to internal sensor 370 located either inside, or on the surface of, container 310, such that internal sensor 370 can obtain information regarding the environment internal to container 310. Controller 350 and internal sensor 360 can take the form of electronic components such as a micro-controller and an electronic sensor. However, the controller-sensor combination may also be chemical or organic materials which perform the same function, such as a liquid crystal sensor/display.

When the shipper or user of container 310 desires information from internal sensor 370 the shipper or user can use reader 340 to query internal sensor 370. In the electronic component embodiment, reader 340 could be a user interface device such as a computer which can be electronically connected to internal sensor 370.

In an additional embodiment, controller 350 can modify the operation of container 310 so as modify the preservation capabilities of container 310, so as to favorably influence a ΔN of the nutritional substance. For example, if the interior environment of container 310 would adversely affect the nutritional substance 320, container 310 could adjust the internal environment of container 310 to better preserve the nutritional substance. If nutritional substance needs to be kept within a certain temperature range to preserve its organoleptic and/or nutritional properties, and the internal sensor 370 provide internal temperature information to controller 350, controller 350 could modify container 310 so as to maintain nutritional substance 320 within the required temperature range.

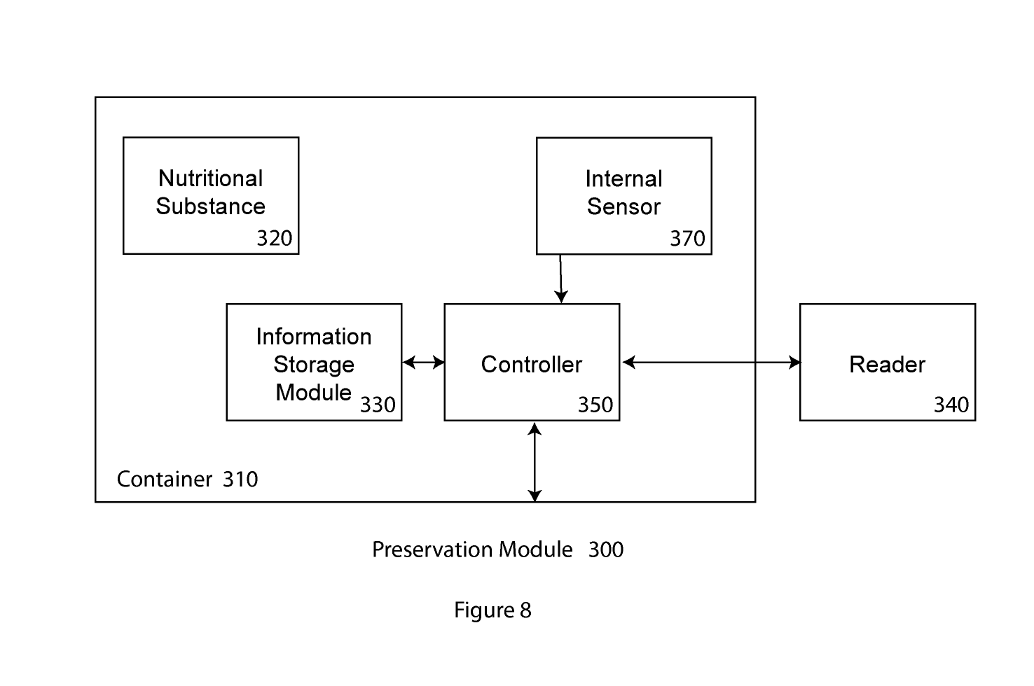

In FIG. 8, preservation system 300 includes container 310 which contains nutritional substance 320, controller 350, and information storage module 330. Internal sensor 370 is positioned such that it can provide information on the internal environment to container 310. Information from the internal sensor and information storage module can be retrieved by connecting reader 340 to container 310.

In this embodiment, information regarding the internal environment sensed by internal sensor 370 and provided to controller 350 can be stored in information storage module 330. This storage of internal environment can be used to record a history the internal environment container 310 has been subjected to. This would allow the shipper or user of container 310 to understand the internal environment the container has been subjected to during the time it has preserved the nutritional substance. Such information can be used to determine any number of ΔN values of the nutritional substance and if the nutritional substance is no longer safe for consumption or has been degraded such that the nutritional substance is no longer in an optimal state. Additionally, the user of the nutritional substance could modify its transformation, conditioning, or consumption according to any changes, or ΔNs, that may have occurred because of the internal conditions of the container.

Additionally, in this embodiment, information storage module 340 could contain other information regarding the nutritional substance, including creation information, identification information, and/or prior transformation information.

In an additional embodiment, controller 350 can modify the operation of container 310 so as modify the preservation capabilities of container 310. For example, if the internal environment of container 310 would adversely affect the nutritional substance 320, container 310 could adjust the internal environment of container 310, so as to favorably influence a ΔN of the nutritional substance. For example, if the internal environment of container 310 would adversely affect the nutritional substance 320, container 310 could adjust the internal environment of container 310 to better preserve the nutritional substance. Controller 350 can analyze the historic information from internal sensor 370, stored in information storage module 330 to determine any long-term internal conditions environmental If nutritional substance needs to be kept within a certain temperature range to preserve its organoleptic and/or nutritional properties, and the internal sensor 370 provide internal temperature information to controller 350, controller 350 could modify container 310 so as to maintain nutritional substance 320 within the required temperature range.

Information in the information storage module 320 might include identification information, information regarding prior transformation of the nutritional substance 320, and other historic information. A shipper, or user, of container 310 can operatively connect to information storage module 330 using reader 340 to retrieve information stored therein. In an alternate embodiment reader 340 can also write to information storage module 330. In this embodiment, information regarding the container and/or nutritional substance 320 can be modified or added to information storage module 330 by the user or shipper.

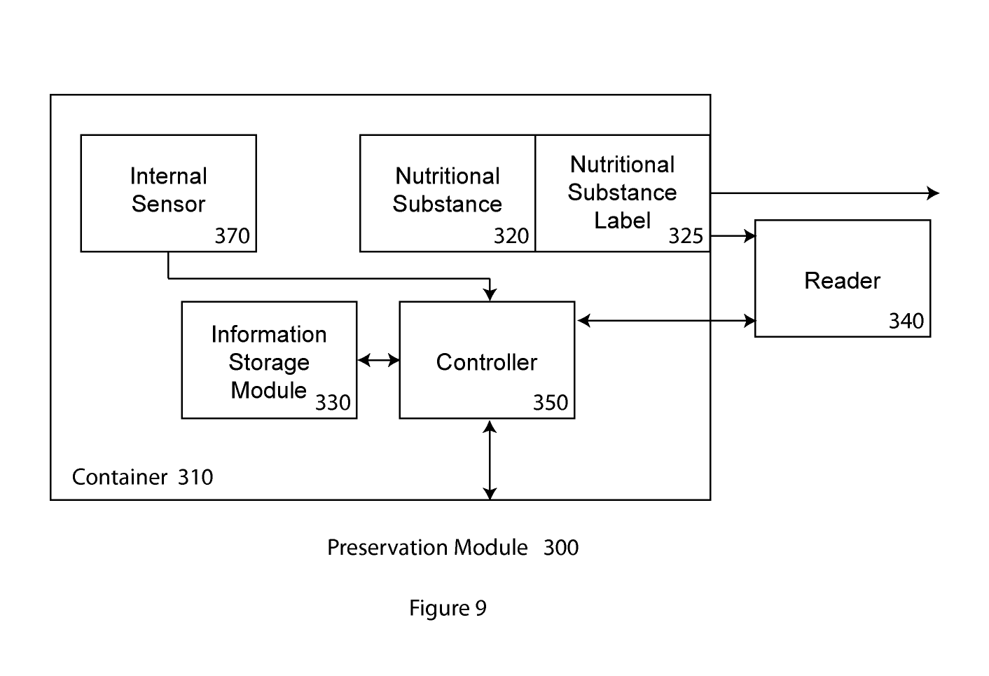

FIG. 9 shows an alternate embodiment of the present invention. Preservation system 300 includes container 310 which contains nutritional substance 320, nutritional substance label 325, controller 350, and information storage module 330. Internal sensor 370 is positioned such that it can provide information on the internal environment to container 310. Information from the internal sensor and information storage module can be retrieved by connecting reader 340 to container 310. Nutritional substance label 325 is attached to nutritional substance 320 so as to sense, measure, and/or indicate the current state of nutritional substance 320. Nutritional substance label 325 can be read by reader 340. Nutritional substance label 325 could be a material/chemical tag that, through a physical reaction with the surface of nutritional substance 320, provides information regarding the nutritional, organoleptic, and aesthetic state of the nutritional substance, or information regarding changes in the nutritional, organoleptic, and aesthetic values of the nutritional substance, including where nutritional substance 320 is in its life cycle. As an example, this label/tag could change color as a fruit, cheese or wine matures across time. It could also indicate if it detects traces of pesticides, hormones, allergens, harmful or dangerous bacteria, or any other substances.

In this embodiment, information regarding the internal environment sensed by internal sensor 370 and provided to controller 350 can be stored in information storage module 330. This storage of internal environment can be used to record a history the internal environment container 310 has been subjected to. This would allow the shipper or user of container 310 to understand the internal environment the container has been subjected to during the time it has preserved the nutritional substance. Such information can be used to determine any number of ΔN values for the nutritional substance, including if the nutritional substance is no longer safe for consumption or has been degraded such that the nutritional substance is no longer in an optimal state. Additionally, the user of the nutritional substance could modify its transformation, conditioning, or consumption according to any changes, or ΔNs, that may have occurred because of the internal conditions of the container.

Additionally, in this embodiment, information storage module 340 could contain other information regarding the nutritional substance, including creation information, identification information, and/or prior transformation information.

In an additional embodiment, controller 350 can modify the operation of container 310 so as modify the preservation capabilities of container 310, so as to favorably influence a ΔN of the nutritional substance. For example, if the internal environment of container 310 would adversely affect the nutritional substance 320, container 310 could adjust the internal environment of container 310 to better preserve the nutritional substance. Controller 350 can analyze the historic information from internal sensor 370, stored in information storage module 330 to determine any long-term internal conditions environmental If nutritional substance needs to be kept within a certain temperature range to preserve its organoleptic and/or nutritional properties, and the internal sensor 370 provide internal temperature information to controller 350, controller 350 could modify container 310 so as to maintain nutritional substance 320 within the required temperature range.

Information in the information storage module 320 might include identification information, information regarding prior transformation of the nutritional substance 320, and other historic information. A shipper, or user, of container 310 can operatively connect to information storage module 330 using reader 340 to retrieve information stored therein. Additionally, such a shipper, or user, of container 310 can obtain information from nutritional substance label 325, either through direct observation or through reader 340. In an alternate embodiment reader 340 can also write to information storage module 330. In this embodiment, information regarding the container and/or nutritional substance 320 can be modified or added to information storage module 330 by the user or shipper.

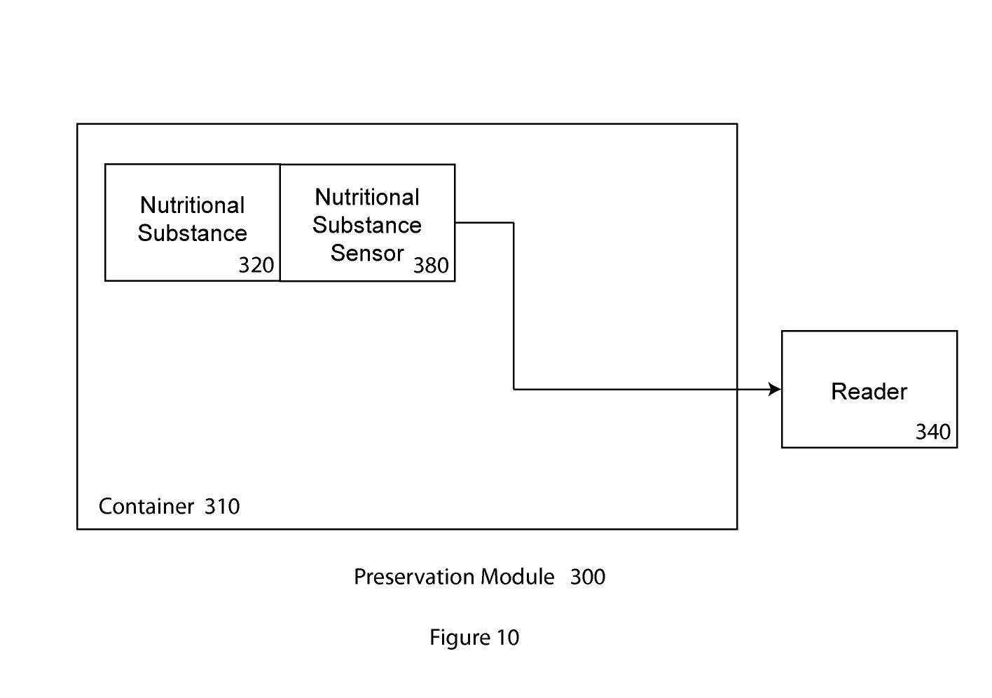

FIG. 10 shows embodiment of preservation system 300 wherein container 310 contains nutritional substance 320 as well as nutritional substance sensor 380 in contact with nutritional substance 320, such that nutritional substance sensor 380 can obtain information regarding the nutritional substance 320 in container 310. Nutritional substance sensor 380 can be connected to reader 340 to obtain the nutritional substance 320 condition. Nutritional substance sensor 380 and reader 340 can take the form of electronic components such as an electronic sensor and electronic display. However, the controller-sensor combination may also be chemical or organic materials which perform the same function, such as a liquid crystal sensor/display.

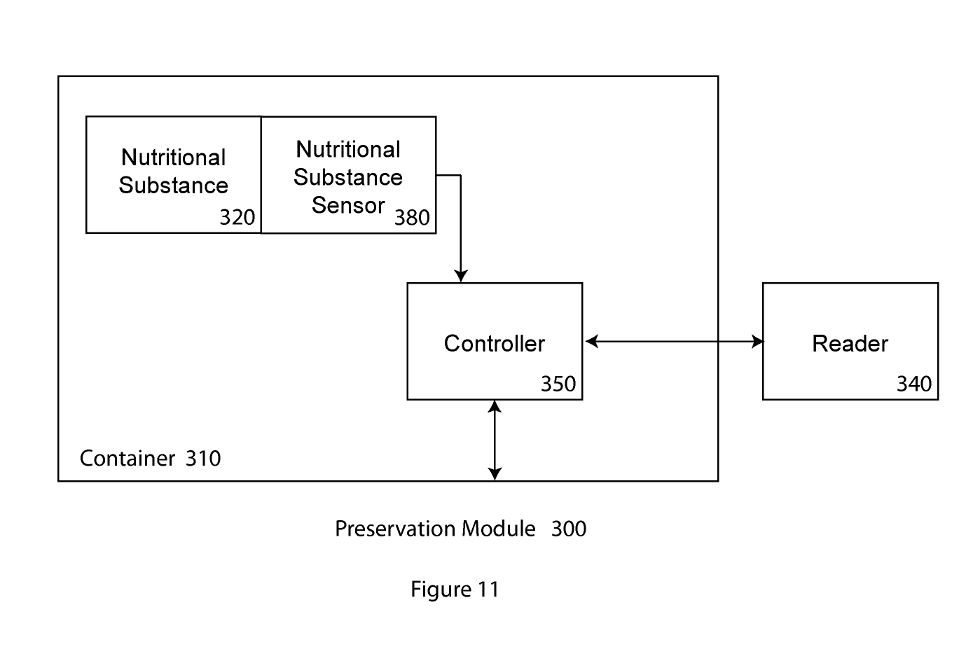

FIG. 11 shows embodiment of preservation system 300 wherein container 310 contains nutritional substance 320 as well as controller 350. Controller 350 is connected to nutritional substance sensor 380. Controller 350 and nutritional substance sensor 380 can take the form of electronic components such as a micro-controller and an electronic sensor. However, the controller-sensor combination may also be chemical or organic materials which perform the same function, such as a liquid crystal sensor/display.

When the shipper or user of container 310 desires information from nutritional substance sensor-380 the shipper or user can use reader 340 to query nutritional substance sensor 380. In the electronic component embodiment, reader 340 could be a user interface device such as a computer which can be electronically connected to nutritional substance sensor 380.

In an additional embodiment, controller 350 can modify the operation of container 310 so as modify the preservation capabilities of container 310, so as to favorably influence a ΔN of the nutritional substance. For example, if the interior environment of container 310 is adversely affecting the nutritional substance 320, container 310 could adjust the nutritional substance environment of container 310 to better preserve the nutritional substance. If nutritional substance needs to be kept within a certain temperature range to preserve its organoleptic and/or nutritional properties, and the nutritional substance sensor 380 provide nutritional substance temperature information to controller 350, controller 350 could modify container 310 so as to maintain nutritional substance 320 within the required temperature range.

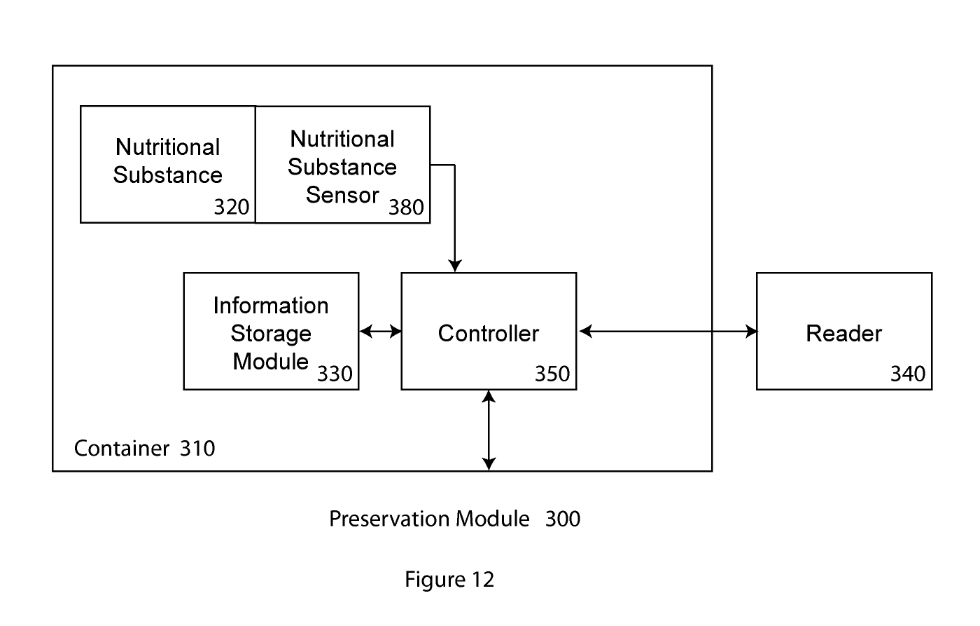

In FIG. 12, preservation system 300 includes container 310 which contains nutritional substance 320, controller 350, and information storage module 330. Nutritional substance sensor 380 is positioned such that it can provide information on the nutritional substance in container 310. Information from the nutritional substance sensor 380 and information storage module can be retrieved by connecting reader 340 to controller 350.

In this embodiment, information regarding the nutritional substance sensed by nutritional substance sensor 380, and provided to controller 350, can be stored in information storage module 330. This storage of nutritional substance environment can be used to record a history the nutritional substance. This would allow the shipper or user of container 310 to understand the nutritional substance during the time it has preserved the nutritional substance. Such information can be used to determine any number of ΔN values of the nutritional substance and if the nutritional substance is no longer safe for consumption or has been degraded such that the nutritional substance is no longer in an optimal state. Additionally, the user of the nutritional substance could modify its transformation, conditioning, or consumption according to any changes, or ΔNs, that may have occurred because of the conditions of the container.

Additionally, in this embodiment, information storage module 330 could contain other information regarding the nutritional substance, including creation information, identification information, and/or prior transformation information.

In an additional embodiment, controller 350 can modify the operation of container 310 so as modify the preservation capabilities of container 310. For example, if the nutritional substance 320 is being adversely affected, controller 350 could adjust the container 310 to better preserve the nutritional substance. Controller 350 can analyze the historic information from nutritional substance sensor 380 stored in information storage module 330 to determine any long-term nutritional substance conditions that need to be changed, If nutritional substance needs to be kept within a certain temperature range to preserve its organoleptic and/or nutritional properties, and the nutritional substance sensor 380 provide nutritional substance temperature information to controller 350, controller 350 could modify container 310 so as to maintain nutritional substance 320 within the required temperature range.

Information in the information storage module 320 might include identification information, information regarding prior transformation of the nutritional substance 320, and other historic information. A shipper, or user, of container 310 can operatively connect to information storage module 330 using reader 340 to retrieve information stored therein. In an alternate embodiment reader 340 can also write to information storage module 330. In this embodiment, information regarding the container and/or nutritional substance 320 can be modified or added to information storage module 330 by the user or shipper.

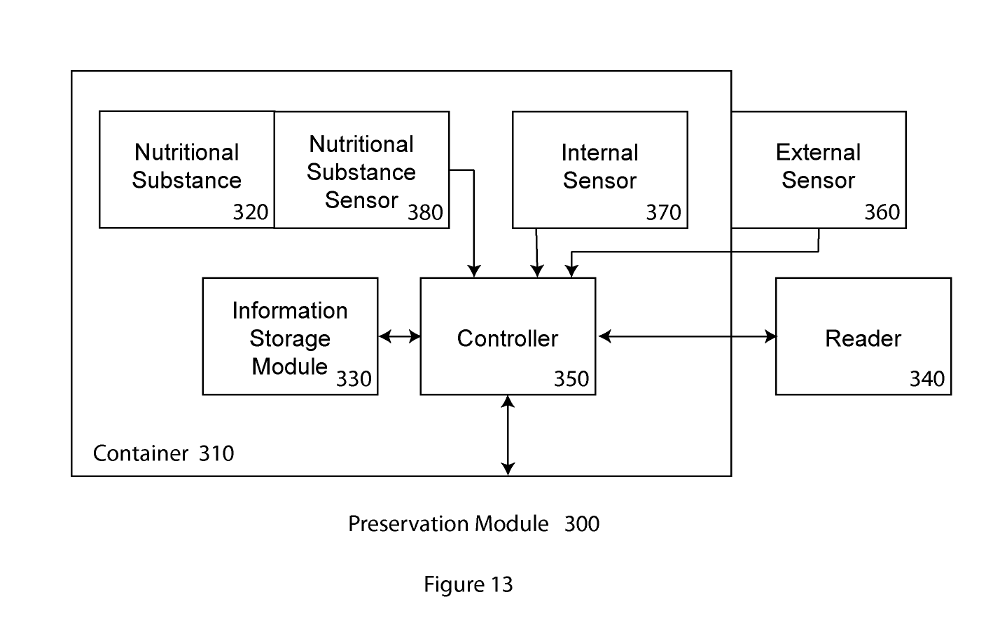

FIG. 13 shows the preferred embodiment of preservation module 300. Within container 310 is nutritional substance 320, nutritional substance sensor 380, internal sensor 370, information storage module 340, and controller 350. External sensor 360 is located outside or on the surface of container 310. In operation, controller 350 receives information from nutritional substance sensor 380, internal sensor 370, and external sensor 360. Additionally, controller 350 can store the information received from the three sensors in information storage module 330. Controller 350 can retrieve such stored information and transmit it to reader 340. Reader 340 can also transmit instructions to controller 350.

Controller 350 is operably connected to container 310 so as to use the information obtained from the sensors and/or information stored in the information storage module to modify the operation of container 310 to affect the state of nutritional substance 320, that is, to favorably influence a ΔN for the nutritional substance. Additionally, storage module 330 could contain information regarding nutritional substance 320 as to its identity, creation information and/or prior transformation information. This historic information could also be used in modifying the operation of container 310 in its preservation of nutritional substance 320.

As an example, nutritional substance 320 could be bananas being shipped to a distribution warehouse. Bananas are in container 310 which is capable of controlling its internal temperature, humidity, and the level of certain gasses within the container. Creation information as to the bananas is placed in information storage module 330 prior to shipment. During shipment, external sensor 360 measures the temperature and humidity outside container 310. This information is stored by controller 350 in information storage module 330. Controller 350 also receives information on the internal environment within container 310 from internal sensor 370 and stores this information in information storage module 330. This information includes the internal temperature, humidity, and certain gas levels within container 310. Finally, nutritional substance sensor 380, which is attached to the surface of the bananas, provides information as to the state of the bananas to controller 350. This information could include surface temperature, surface humidity, gasses being emitted, and surface chemicals. At any time during its shipment and delivery to the distribution warehouse, reader 340 can be used to retrieve both current information and historic information stored within information storage module 330.

During shipment, container 310 modifies its internal conditions according to instructions provided by controller 350. Controller 350 contains instructions as to how to preserve, and possibly ripen, the bananas using information stored in information storage module 330 about the creation of the bananas, as well as historical information received from the three sensors, as well as current information being received from the three sensors. In this manner, preservation module 300 can preserve and optimize and minimize degradation of the bananas. In other words, preservation module 300 can operate in a way to favorably influence changes in aesthetic, organoleptic and nutritional values/attributes, ΔNs, of the bananas while they are being shipped and stored.

It will be understood that subsets of the embodiment described herein can operate to achieve the goals stated herein. In one embodiment, nutritional substance sensor 380, internal sensor 370, external sensor 360, information storage module 330, controller 350, reader 340, and parts of container 310 are each electrical or electromechanical devices which perform each of the indicated functions. However, it is possible for some or all of these functions to be done using chemical and/or organic compounds. For example, a specifically designed plastic wrap for bananas can sense the exterior conditions of the package, the interior conditions of the package, and control gas flow through its surface so as to preserve and ripen the bananas.

Unless the context clearly requires otherwise, throughout the description and the claims, the words “comprise,” “comprising,” and the like are to be construed in an inclusive sense (i.e., to say, in the sense of “including, but not limited to”), as opposed to an exclusive or exhaustive sense. As used herein, the terms “connected,” “coupled,” or any variant thereof means any connection or coupling, either direct or indirect, between two or more elements. Such a coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the invention is not intended to be exhaustive or to limit the invention to the precise form disclosed above. While specific examples for the invention are described above for illustrative purposes, various equivalent modifications are possible within the scope of the invention, as those skilled in the relevant art will recognize. While processes or blocks are presented in a given order in this application, alternative implementations may perform routines having steps performed in a different order, or employ systems having blocks in a different order. Some processes or blocks may be deleted, moved, added, subdivided, combined, and/or modified to provide alternative or sub-combinations. Also, while processes or blocks are at times shown as being performed in series, these processes or blocks may instead be performed or implemented in parallel, or may be performed at different times. Further any specific numbers noted herein are only examples. It is understood that alternative implementations may employ differing values or ranges.

The various illustrations and teachings provided herein can also be applied to systems other than the system described above. The elements and acts of the various examples described above can be combined to provide further implementations of the invention.

Any patents and applications and other references noted above, including any that may be listed in accompanying filing papers, are incorporated herein by reference. Aspects of the invention can be modified, if necessary, to employ the systems, functions, and concepts included in such references to provide further implementations of the invention.

These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

While certain aspects of the invention are presented below in certain claim forms, the applicant contemplates the various aspects of the invention in any number of claim forms. For example, while only one aspect of the invention is recited as a means-plus-function claim under 35 U.S.C. §112, sixth paragraph, other aspects may likewise be embodied as a means-plus-function claim, or in other forms, such as being embodied in a computer-readable medium. Any claims intended to be treated under 35 U.S.C. §112, ¶6 will begin with the words “means for.” Accordingly, the applicant reserves the right to add additional claims after filing the application to pursue such additional claim forms for other aspects of the invention.

I claim:

1. A method of dynamically ascertaining an expiration date for nutritional substances comprising the steps of:
measuring a dynamically changing condition associated with a nutritional substance; and
comparing said measured condition to known conditions associated with similar nutritional substances to determine if said nutritional substance has passed its expiration date.

2. The method of dynamically ascertaining an expiration date for nutritional substances according to claim 1 wherein the measured condition is at least one of a nutritional, organoleptic, and aesthetic attribute of the nutritional substance.

3. The method of dynamically ascertaining an expiration date for nutritional substances according to claim 1 wherein the measured condition is an attribute of the nutritional substance's environment.

4. The method of dynamically ascertaining an expiration date for nutritional substances according to claim 1 wherein the measured condition is an attribute of the nutritional substance's packaging.

5. The method of dynamically ascertaining an expiration date for nutritional substances according to claim 1 wherein the known conditions associated with similar nutritional substances are based on at least one of experimentation and algorithm.

6. A method of dynamically ascertaining an expiration date for nutritional substances according to claim 1 further comprising:
conveying to a consumer if said nutritional substance has passed its expiration date.

7. A method of dynamically ascertaining an expiration date for nutritional substances according to claim 6, wherein
said conveying to a consumer is accomplished by at least one of a language, number, symbol, code, or sound which the consumer can identify.
